(19) United States
(12) Patent Application Publication
(10) Pub. No.: US 2024/0216620 A1
(43) Pub. Date: Jul. 4, 2024

(54) EVAPORATION CHAMBER AND CHANNEL MODULE FOR ACTIVE AEROSOL SUCTION DEVICE

(71) Applicants: Yun-Shan CHANG, San Jose, CA (US); Da-Wei LIN, Taichung City (TW)

(72) Inventors: Yun-Shan CHANG, San Jose, CA (US); Da-Wei LIN, Taichung City (TW)

(21) Appl. No.: 18/192,013

(22) Filed: Mar. 29, 2023

(30) Foreign Application Priority Data
Jan. 4, 2023 (TW) 112100262

(51) Int. Cl. A61M 11/00; A61M 11/04

(52) U.S. Cl. CPC A61M 11/005; A61M 11/003; A61M 11/042; A61M 2205/3334

(57) ABSTRACT

The invention discloses a method and a device for producing a medicine particle flow, belonging to medical health care instruments. The device is characterized by comprising a water mist production device equipped with an ultrasonic atomization device and a fan, and a first closed channel equipped with a heating device, wherein a water mist guide outlet of the water mist production device is connected with a water mist inlet of the first closed channel. Compared with the prior art, the method and the device of the invention have the advantage that effective components of Chinese herbal medicines without volatility can reach the skin together with steam in the form of fine particles, or even molecules, so as to be conveniently absorbed by the skin.

### BACKGROUND OF THE INVENTION

### 1. Field of the Invention

The present invention relates to an evaporation chamber and channel module, especially to an evaporation chamber and channel module for an active aerosol suction device.

### 2. Description of the Prior Arts

An active aerosol suction device is configured to turn liquid into small molecule clusters by heating, and then a user inhales the small molecule clusters into the respiratory system. The liquid can be medicine capable of easing symptoms or treating diseases related to the respiratory system.

The heating mechanism of the conventional active aerosol suction device is to absorb the liquid through the wick to the heating module first, and then the heating module atomizes the liquid in the wick. Finally the liquid molecule clusters are mixed with the airflow inhaled by the user, and then flow out from the device with the airflow. However, the atomized molecule clusters cannot be mixed with the airflow uniformly, and thus the concentration of the medicine inhaled into the respiratory system of the patient is not uniform. The medicine is not able to arrive at the lower respiratory system of the patient because the medicine molecule clusters are unable to mix uniformly with the airflow, thereby decreasing the healing effect of the medicine.

To overcome the shortcomings, the present invention provides an evaporation chamber and channel module to mitigate or obviate the aforementioned problems.

### SUMMARY OF THE INVENTION

The main objective of the present invention is to provide an evaporation chamber and channel module for an active aerosol suction device that is capable of atomizing liquid into small molecule clusters and then mixing with an airflow.

The evaporation chamber and channel module has a main body, two channels, a first opening, and an atomization unit. A chamber is formed within the main body. Each one of the two channels fluidly communicates with the chamber and the exterior. The first opening is formed on the main body, and the first opening fluidly communicates with the chamber. The atomization unit is mounted in the chamber.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of an evaporation chamber and channel module according to an embodiment in accordance with the present invention;

FIG. 2 is a cross-sectional perspective view of the evaporation chamber and channel module in FIG. 1;

FIG. 3 is a side view of the evaporation chamber and channel module in FIG. 2;

FIG. 4 is a partial enlarged view of a capillary channel of the evaporation chamber and channel module in FIG. 3;

FIG. 5 is a cross-section perspective view of an evaporation chamber and channel module according to an alternative embodiment;

FIG. 6 is a partial enlarged cross-section view of the one-way valve which is closed in the evaporation chamber and channel module in FIG. 5;

FIG. 7 is a partial enlarged cross-section view of the one-way valve which is open in the evaporation chamber and channel module in FIG. 5.

### DETAILED DESCRIPTION OF THE PREFERRED EMBODIMENTS

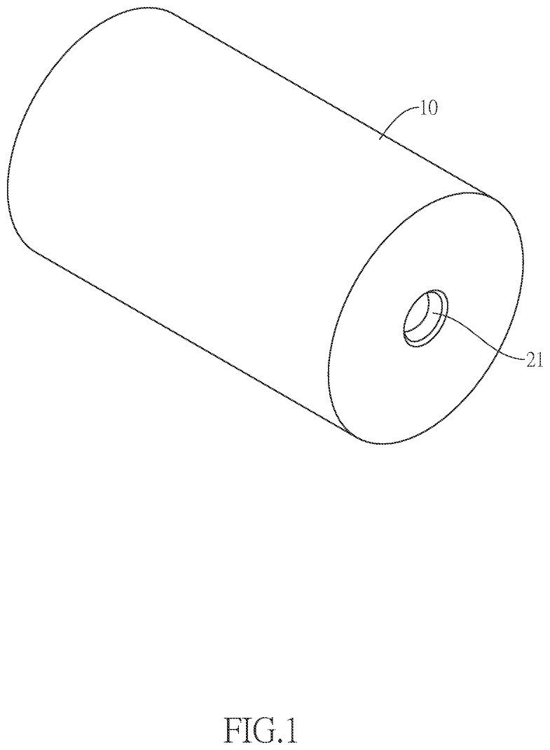

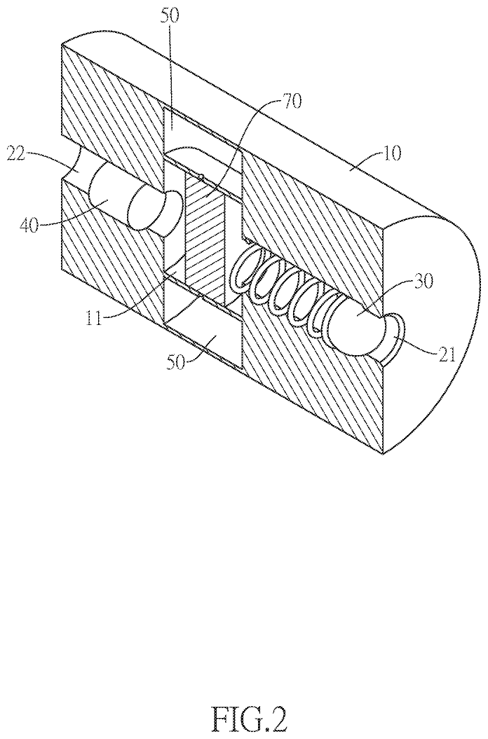

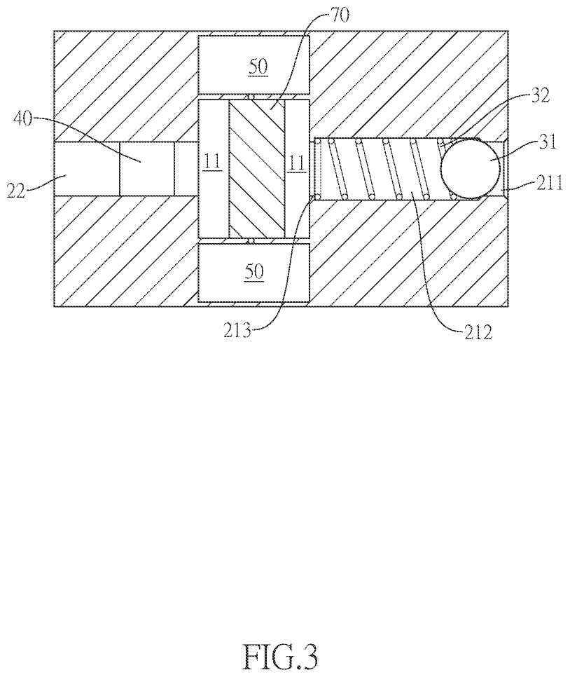

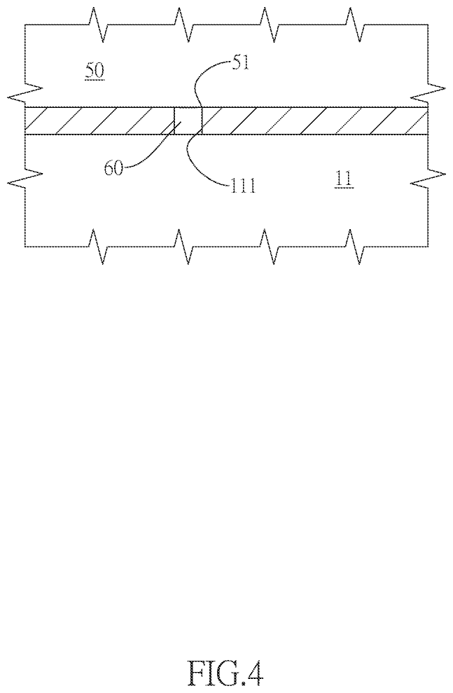

With reference to FIGS. 1 to 4, an evaporation chamber and channel module in accordance with the present invention is configured to make liquid turn into numerous small molecule clusters and distribute the molecule clusters in a space, and then the evaporation chamber and channel module mixes the molecule clusters with the airflow.

The first embodiment of the evaporation chamber and channel module includes a main body 10, two channels, a one-way valve 30, a filter 40, a liquid vessel 50, a capillary channel 60, and an atomization unit 70.

A chamber 11 is formed in the main body 10. The two channels are formed in the main body 10 and the both channels fluidly communicate with an exterior of the main body 10 and the chamber 11. The two channels includes one input channel 21 and one output channel 22. The input channel 21 includes an entering section 211, a connecting section 212, and an abutting unit 213. The entering section 211 fluidly communicates with the exterior of the main body 10. The connecting section 212 fluidly communicates with the chamber 11 and the entering section 211. A diameter of the connecting section 212 is larger than a diameter of the entering section 211. The abutting unit 213 is formed at an end of the connecting section 212, and the end of the connecting section 212 fluidly communicates with the chamber 11. The abutting unit 213 protrudes inward from an inner wall of the connecting section 212.

The one-way valve 30 is mounted in the input channel 21. The one-way valve 30 may include a blocking unit 31 and an elastic unit 32. The blocking unit 31 is mounted in the connecting section 212, the blocking unit 31 selectively abuts a surface of the entering section 211, and the surface of the entering section 211 faces the connecting section 212. A diameter of the blocking unit 31 is larger than the diameter of the entering section 211, and the diameter of the blocking unit 31 is smaller than the diameter of the connecting section 212. The elastic unit 32 is disposed in the connecting section 212. An end of the elastic unit 32 is connected to the blocking unit 31, and the other end of the elastic unit 32 is connected to the abutting unit 213. Therefore, the elastic unit 32 would not enter the chamber 11.

The filter 40 is mounted in the output channel 22. The filter 40 is configured to absorb larger droplets of the atomized liquid when the atomized liquid goes out of the chamber 11 with the airflow. Therefore, the filter 40 is capable of preventing the user from inhaling the larger droplets. In this embodiment, the filter 40 contains porous materials, a diameter of the pores is related to the ingredients of the liquid. In another embodiment, the filter 40 may be a micro pump.

The first opening 111 is formed in the main body 10, and the first opening 111 fluidly communicates with the chamber 11. The liquid vessel 50 is mounted beside the chamber 11, and a second opening 51 is disposed on the liquid vessel 50. The evaporation chamber and channel module in this embodiment further has a capillary channel 60. The capillary channel 60 fluidly communicates with the first opening 111 and the second opening 51, and thus the capillary channel 60 fluidly communicates with the chamber 11 and the liquid vessel 50. To describe more precisely, a capillary action may occur in the capillary channel 60, thereby causing the liquid in the liquid vessel 50 to flow into the chamber 11. In another embodiment, there may be no capillary channel 60 between the chamber 11 and the liquid vessel 50.

The atomization unit 70 is mounted in the chamber 11. The atomization unit 70 is a heating unit in this embodiment. In another embodiment, the atomization unit 70 may be an ultrasound vibration unit.

When the evaporation chamber and channel module is working, the liquid in the liquid vessel 50 flows into the chamber 11 through the capillary channel 60 which fluidly communicates with the second opening 51 and the first opening 111. The liquid gets atomized and forms numerous small molecule clusters after receiving heat from the atomization unit 70. Because the gas in the chamber is in a quasi-steady state and achieves dynamic equilibrium in thermodynamics, the molecule clusters are uniformly formed in the chamber 11.

Next, a user creates low pressure in the chamber 11 by suctioning air with a pump or inhaling air by mouth through the output channel 22, then airflow enters the chamber 11 through the entering section 211 of the input channel 21. The airflow pushes the blocking unit 31 and presses the elastic unit 32, and thereby a gap is formed between the blocking unit 31 and the surface which faces the connecting section 212 of the entering section 211, and finally the airflow enters the connecting section 212. The airflow then flows into the chamber 11, receives heat from the atomization unit 70, mixes with the atomized liquid, and finally flows out through the output channel 22.

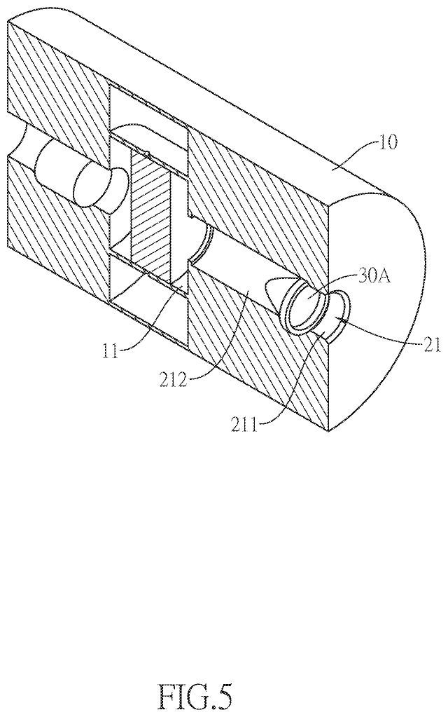

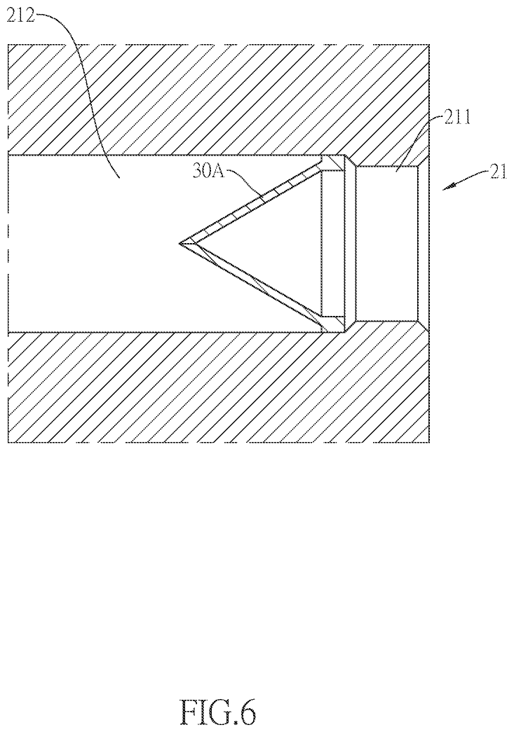

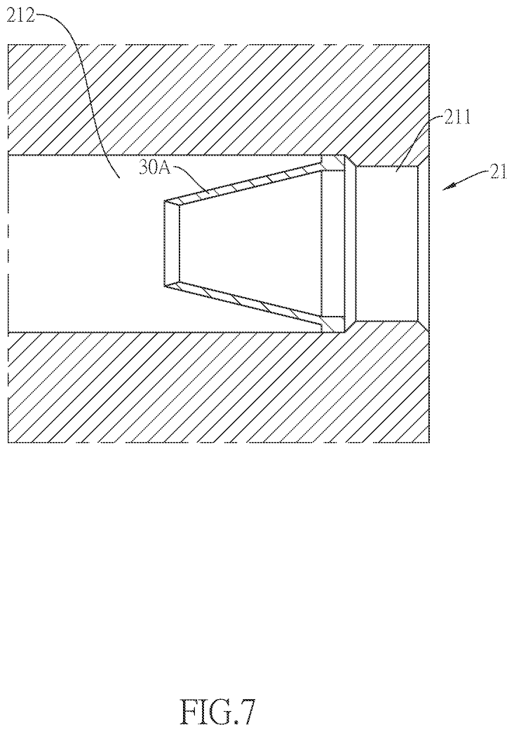

With reference to FIGS. 5 to 7, the second embodiment of this invention has a one-way valve 30A in a different configuration. The one-way valve 30A is mounted in the connecting section 212 of the input channel 21. The one-way valve 30A includes a membrane unit. The membrane unit is in the shape of a cone. The one-way valve 30A is mounted on the surface of the entering section 211, and the surface faces the connecting section 212. The vertex of the one-way valve 30A is oriented toward the chamber 11.

An airflow inlet is formed at the vertex of the membrane unit, and the airflow inlet is adapted to be selectively opened and closed. To describe more precisely, when the airflow is passing through the one-way valve 30A, the airflow inlet is opened up by the airflow, and the airflow flows into the connecting section 212; when the airflow stops flowing, and the airflow inlet closes up, thereby preventing the gas in the chamber 11 from leaking out from the input channel 21.

In summary, the evaporation chamber and channel module in this invention is capable of atomizing the liquid with the atomization unit 70 in the chamber 11, mixing the liquid molecule clusters with the airflow. The one-way valve 30 mounted in the input channel 21 is capable of preventing the air leaking through the input channel 21. The filter 40 is capable of preventing the user inhaling the larger droplets resulting from incomplete atomization by absorbing them in the output channel 22.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. An evaporation chamber and channel module configured to atomize liquid and mix the liquid with an airflow, the evaporation chamber and channel module comprising:
a main body, and a chamber formed within the main body;
two channels, each one of the channels fluidly communicating with the chamber and an exterior of the main body;
a first opening formed on the main body, the first opening fluidly communicating with the chamber; and
an atomization unit mounted in the chamber.

2. The evaporation chamber and channel module as claimed in claim 1, wherein the evaporation chamber and channel module further comprises:
a liquid vessel disposed beside the chamber, the liquid vessel including a second opening, and the first opening fluidly communicating with the second opening.

3. The evaporation chamber and channel module as claimed in claim 2, wherein the evaporation chamber and channel module further comprises:
a capillary channel fluidly communicating with the first opening and the second opening.

4. The evaporation chamber and channel module as claimed in claim 1, wherein the two channels include an input channel, and a one-way valve mounted at the input channel.

5. The evaporation chamber and channel module as claimed in claim 3, wherein the two channels include an input channel, and a one-way valve mounted at the input channel.

6. The evaporation chamber and channel module as claimed in claim 4, wherein:
the input channel comprises:
an entering section fluidly communicating with the exterior of the main body;
a connecting section fluidly communicating with the chamber and the entering section, and a diameter of the connecting section being larger than a diameter of the entering section; and
an abutting unit formed at an end of the connecting section, the end of the connecting section fluidly communicating with the chamber; the abutting unit protruding inward from an inner wall of the connecting section; and
the one-way valve comprises:
a blocking unit mounted in the connecting section, the blocking unit selectively abutting a surface of the entering section, the surface of the entering section facing the connecting section; a diameter of the blocking unit being larger than the diameter of the entering section, and the diameter of the blocking unit being smaller than the diameter of the connecting section; and
an elastic unit mounted in the connecting section, one end of the elastic unit connected to the blocking unit and another end of the elastic unit connected to the abutting unit.

7. The evaporation chamber and channel module as claimed in claim 5, wherein:
the input channel comprises:
an entering section fluidly communicating with the exterior of the main body;
a connecting section fluidly communicating with the chamber and the entering section, and a diameter of the connecting section being larger than a diameter of the entering section; and
an abutting unit formed at an end of the connecting section, the end of the connecting section fluidly communicating with the chamber; the abutting unit protruding inward from an inner wall of the connecting section; and
the one-way valve comprises:
a blocking unit mounted in the connecting section, the blocking unit selectively abutting a surface of the entering section, the surface of the entering section facing the connecting section; a diameter of the blocking unit being larger than the diameter of the entering section, and the diameter of the blocking unit being smaller than the diameter of the connecting section; and
an elastic unit mounted in the connecting section, one end of the elastic unit connected to the blocking unit and another end of the elastic unit connected to the abutting unit.

8. The evaporation chamber and channel module as claimed in claim 4, wherein:
the input channel comprises:
an entering section fluidly communicating with the exterior of the main body; and
a connecting section fluidly communicating with the chamber and the entering section, and a diameter of the connecting section being larger than a diameter of the entering section; and
the one-way valve comprises:
a membrane unit being in the shape of cone, and a vertex of the membrane unit oriented toward the chamber; the membrane unit mounted on a surface of the entering section, the surface facing the connecting section; the membrane unit including:
an airflow inlet formed at the vertex of the membrane unit, the airflow inlet adapted to be selectively opened and closed.

9. The evaporation chamber and channel module as claimed in claim 5, wherein:
the input channel comprises:
an entering section fluidly communicating with the exterior of the main body; and
a connecting section fluidly communicating with the chamber and the entering section, and a diameter of the connecting section being larger than a diameter of the entering section; and
the one-way valve comprises:
a membrane unit being in the shape of cone, and a vertex of the membrane unit oriented toward the chamber; the membrane unit mounted on a surface of the entering section, the surface facing the connecting section; the membrane unit including:
an airflow inlet formed at the vertex of the membrane unit, the airflow inlet adapted to be selectively opened and closed.

10. The evaporation chamber and channel module as claimed in claim 1, wherein the two channels include an output channel, and a filter is mounted in the output channel to absorb droplets of the liquid.

11. The evaporation chamber and channel module as claimed in claim 7, wherein the two channels include an output channel, and a filter is mounted in the output channel to absorb droplets of the liquid.

12. The evaporation chamber and channel module as claimed in claim 9, wherein the two channels include an output channel, and a filter is mounted in the output channel to absorb droplets of the liquid.

13. The evaporation chamber and channel module as claimed in claim 10, wherein the filter includes porous filter or a micro pump.

14. The evaporation chamber and channel module as claimed in claim 11, wherein the filter includes porous filter or a micro pump.

15. The evaporation chamber and channel module as claimed in claim 12, wherein the filter includes porous filter or a micro pump.

16. The evaporation chamber and channel module as claimed in claim 1, wherein the atomization unit includes a heating unit or an ultrasound vibration unit.

17. The evaporation chamber and channel module as claimed in claim 14, wherein the atomization unit includes a heating unit or an ultrasound vibration unit.

18. The evaporation chamber and channel module as claimed in claim 15, wherein the atomization unit includes a heating unit or an ultrasound vibration unit.
